(19) United States
(12) Patent Application Publication
(10) Pub. No.: US 2026/0102452 A1
(43) Pub. Date: Apr. 16, 2026

(54) NANOEMULSION-BASED NANOCREAM PRODUCT FROM GREEN INGREDIENTS EXHIBITING ENHANCED PERFORMANCE CHARACTERISTICS

(71) Applicants: Daphne HT Nguyen, San Jose, CA (US); Manish Kumar, Pindwara (IN)

(72) Inventors: Daphne HT Nguyen, San Jose, CA (US); Manish Kumar, Pindwara (IN)

(21) Appl. No.: 18/915,367

(22) Filed: Oct. 15, 2024

(51) Int. Cl. A61K 36/54; A61K 9/06; A61K 9/107; A61K 36/185; A61K 36/534; A61K 47/10; A61K 47/14; A61K 47/26; A61K 47/44

(52) U.S. Cl. CPC A61K 36/54; A61K 9/06; A61K 9/1075; A61K 36/185; A61K 36/534; A61K 47/10; A61K 47/14; A61K 47/26; A61K 47/44

(57) ABSTRACT

The invention introduces a nanoemulsion-based nanocream formulated from green ingredients, designed to improve performance, stability, and sustainability. The nanoemulsion is developed using the low-energy Phase Inversion Temperature (PIT) method, which leverages the system's internal chemical energy to minimize external energy input, protect heat-sensitive compounds, and lower production costs. During PIT, polyethoxylated surfactants, hydrophilic at lower temperatures, become lipophilic when heated as their polyoxyethylene groups dehydrate, triggering phase inversion from oil-in-water (O/W) to water-in-oil (W/O). Rapid cooling with continuous stirring reverts to the O/W phase, causing turbulence and generating a stable nanoemulsion with nano-sized droplets of high uniformity. This nanoemulsion replaces water in the cream's aqueous phase, forming a nanocream that enhances bioactive delivery to the skin. The cream's thick, highly viscous oily base minimizes the risk of coalescence and further improves the overall stability of the nano-sized droplets containing bioactive oils.

### FIELD OF INVENTION

The present invention provides a composition and method to formulate a nanoemulsion-based nanocream for topical application. The method comprises the formulation of a nanoemulsion by a low-energy emulsification technique wherein the green bioactive compounds are protected from degradation. The low-energy emulsification method reduces the need for high-energy input thereby also lowering preparation costs. The said nanoemulsion developed by the low-energy emulsification technique is utilized in the formulation of the nanocream product that exhibits enhanced performance characteristics and improved stability.

### BACKGROUND OF THE INVENTION

The following description of the related art is intended to provide background information pertaining to the field of the disclosure. This section may include certain aspects of the art that may be related to various features of the present disclosure. However, it should be appreciated that this section is used only to enhance the understanding of the reader with respect to the present disclosure, and not as admissions of the prior art.

Nanoemulsions are stable mixtures of two immiscible liquids, typically oil and water. Herein, one liquid is dispersed in the other liquid in the form of tiny droplets usually having either O/W (oil-in-water) composition or W/O (water-in-oil) composition. The term ‘nano’ in nanoemulsions refers to the tiny droplets, which range in size from 20 to 200 nanometers. The small size of these droplets provides a larger surface area thereby enhancing the stability, solubility, and bio-availability of the active compounds. Due to these characteristics, nanoemulsions are widely used in various applications including pharmaceuticals, cosmetics, food products, and even agrochemicals.

The formation of nanoemulsions involves the selection of ideal components for the oily phase, the aqueous phase, and the surfactants. The selected oil phase is combined with the surfactants using techniques like high-shear mixing, ultrasonication, or micro-fluidization to create small droplets. By applying mechanical forces like high-pressure homogenizers or ultrasonic homogenizers, the size of the droplets is reduced. The presence of the surfactants, which adsorb onto the surface of the droplets, provides stability and prevents coalescence.

Nanoemulsions have a wide range of applications including drug delivery, vaccines, cosmetics, skincare, creating emulsified products in the food industry, and paints and coatings. In recent developments, topical applications of nanoemulsions are gaining popularity, due to their capabilities of efficient drug delivery, improved skin penetration properties, and controlled release.

However, there are several drawbacks associated with the traditional techniques of formulating nanoemulsions. Traditional techniques such as high-pressure homogenization and ultrasonication require significant energy input that could degrade the sensitive compounds and also lead to higher production costs. In addition, the use of synthetic surfactants in many traditional techniques poses safety concerns as they can lead to potential toxicity and allergies. Furthermore, the nanoemulsions from traditional techniques suffer from low stability and low viscosity that doesn't meet the requirement of the required texture. Therefore, the nanoemulsions obtained from traditional techniques have limited therapeutic applications and performance. The low-energy emulsification method, and the corresponding nanocream formulation, can overcome these limitations by offering several advantages over high-energy techniques, including protecting heat-sensitive bioactives and offering lower preparation costs. Nanocream formulation containing bioactives at the nanometer scale can be used in topical applications to cater to various needs, including hydration, protective barriers, and imparting therapeutic effects.

The present invention deals with the formulation of a nanoemulsion-based nanocream product for topical application to leverage improved performance characteristics. The invention replaces traditional phases of the nanocream base with the nanoemulsion, thereby enhancing the nanocream's stability.

### OBJECTIVES OF THE INVENTION

1. The primary objective of the present invention is to formulate a nanoemulsion-based nanocream product from green ingredients derived from renewable resources, such as plants, minerals, or bacteria.
2. The other objective of the present invention is to formulate a nanoemulsion with a low-energy method and improve its stability.
3. The other objective of the present invention is to lower the production cost of the nanocream product.
4. The other objective of the present invention is to mitigate the risk of damaging sensitive bioactive compounds.
5. The other objective of the present invention is to avoid the problems of poor productivity and heat-induced deterioration of the compounds.
6. The other objective of the present invention is to replace the aqueous phase of the nanocream with the nanoemulsion to improve its performance characteristics
7. The other objective of the present invention is to improve the stability of the nanocream product

Other objects and advantages of the present invention will become apparent from the following description taken in connection with the accompanying drawings, wherein, by way of illustration and example, the aspects of the present invention are disclosed.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

The present invention relates to a nanoemulsion-based nanocream product developed by relying on green ingredients. The green ingredient refers to a natural or naturally derived non-toxic substance used in cosmetic or skin care formulations, obtained from renewable resources like plants, minerals, or bacteria. It is processed through eco-friendly methods that preserve its natural integrity and sustainability.

In accordance with the present invention, the nanoemulsion is developed by low-energy emulsification such as the Phase Inversion Temperature (PIT) method, which harnesses the system's internal chemical energy. The method helps lower the preparation loss, promotes sustainability, and reduces the risk of damage to sensitive compounds like proteins and peptides.

As per the first embodiment of the present invention a nanoemulsion for the formulation of a nanocream product comprising an aqueous phase, an oil phase comprising oil or a blend of oils having one or more hydrophobic active ingredients, a primary surfactant system having at least one ionic and at least non-ionic part, and a co-surfactant system wherein glycerine, cetostearyl alcohol, and glyceryl monostearate act as stabilizers.

Further, in the first embodiment, the blend of oils in the oil phase comprises 10-50% of tocopherol, 10-50% cinnamomum oil, 10-50% simmondsia oil, 10-50% mentha oil, the primary surfactant including but not limited to Tween 80, Tween 20, sorbitan monooleate, polyglycerol esters, cocamidopropyl betaine, and the co-surfactant including but not limited to glycerine, cetosterearyl alcohol, and glyceryl monostearate.

As per the second embodiment of the present invention, the nanoemulsion for the formulation of a nanocream product is developed by using the phase inversion temperature method. In this method, polyethoxylated surfactants, such as Tween 80, which are hydrophilic at lower temperatures, typically form oil-in-water (O/W) emulsions. However, when the emulsion system is heated, the polyoxyethylene groups in these surfactants undergo dehydration, making the surfactants more lipophilic. As a result, the spontaneous curvature of the surfactant molecules shifts, leading to the formation of a water-in-oil (W/O) emulsion. The temperature at which this phase inversion occurs is referred to as the Phase Inversion Temperature (PIT), also known as the HLB (Hydrophilic-Lipophilic Balance) temperature.

To produce a nanoemulsion using the Phase Inversion Temperature (PIT) method, the oil-in-water O/W emulsion system is heated until the phase inversion occurs. Subsequently, the emulsion system is rapidly cooled with continuous stirring, a process called temperature quenching. This cooling reverses the phase inversion, transforming the system back to an oil-in-water O/W emulsion. The turbulence caused by the rapid temperature change promotes the formation of nano-sized oil droplets, resulting in a stable oil-in-water O/W nanoemulsion.

As per the third embodiment of the present invention, the formulated oil-in-water nanoemulsion replaces the water in the aqueous phase during the formulation of the nanocream product. The replacement of the water with the nanoemulsion in the aqueous phase of the nanocream improves the stability of the nano-sized droplets and reduces the risk of coalescence. For the oil phase of the nanocream product, natural emulsifiers and waxes including but not limited to beeswax, cocoa butter, lecithin, agar, xanthan gum, palm wax, rice bran wax, candelilla wax, carnauba wax, cetostearyl alcohol, glyceryl monostearate, polyglycerol esters, sorbitan monostearate, stearoyl lactylate, monoglycerides, and diglycerides were utilized as the cream's oily base.

As per the fourth embodiment of the present invention, a method to formulate the nanocream product based on the nanoemulsion system has been disclosed wherein the oil phase is prepared by mixing and weighing the oil phase ingredients and emulsifiers and melting the mixture at 40-70° C. while continuously stirring. The aqueous phase is prepared by combining the formulated nanoemulsion and glycerine and heating the combination to 35-55° C. below the phase inversion temperature of the nanoemulsion. Thereafter, the pre-heated aqueous phase is gradually added to the pre-heated oil phase with continuous stirring and ultimately cooling the mixture to room temperature and during this step, the cooling process is conducted slowly to ensure a smooth emulsion. The complete addition of the aqueous phase to the oil phase while continuously stirring and cooling leads to a homogeneous and viscous nanocream product.

Other objects and advantages of the present invention will become apparent from the following description taken in connection with the accompanying drawings, wherein, by way of illustration and example, the aspects of the present invention are disclosed.

### Definitions

1. Green ingredient: A green ingredient is a natural or naturally derived non-toxic substance used in topical formulations, obtained from renewable resources like plants, minerals, or bacteria. It is processed through eco-friendly methods that preserve its natural integrity and sustainability.
2. Phase Inversion Temperature: Phase Inversion Temperature refers to a temperature at which the system undergoes phase change. In the context of emulsions, it is the temperature where continuous phase change from water to oil or from oil to water occurs.
3. Nanocream products: These are the products that have nanosized bioactives dispersed in cream base. It form a protective barrier on the skin upon topical applications, and help the skin to restore important nutrients. Nanocream may include oils of various types including jojoba, coconut, olive, and other mineral oils, fatty acids such as stearic acid and oleic acid, alcohols like cetyl and stearyl alcohol, waxes such as beeswax, lanolin, humectants like glycerine, hyaluronic acid, and several others lubrication products.
4. Nanoemulsion: Nanoemulsions are stable mixtures of two immiscible liquids, typically oil and water. Herein, one liquid is dispersed in the other liquid in the form of tiny droplets exhibiting mostly either O/W oil-in-water composition or W/O water-in-oil composition. The term ‘nano’ in nanoemulsions refers to the tiny droplets, the size of which varies within the range of 20 to 200 nanometers. These tiny droplets' size, provides a larger surface area thereby enhancing the stability, solubility, and bio-availability of the active compounds, due to these characteristics of the nanoemulsions they are widely used in various applications including pharmaceuticals, cosmetics, food products, and even agrochemicals.
5. Surfactant: Surfactants are compounds having both hydrophilic and hydrophobic properties, due to which they reduce the interfacial tension between two substances and stabilize the emulsion. It adsorbs onto the droplet surfaces, providing stabilization and preventing coalescence.
6. Tocopherol: Tocopherol refers to a class of organic chemical compounds exhibiting vitamin E activity. It acts as an antioxidant and protects cells from damage caused by free radicals.
7. Cinnamomum oil: Cinnamomum oil is derived from the bark or leaves of the cinnamon trees and is widely known for its aromatic properties. It has antimicrobial, anti-fungal, and anti-inflammatory properties.
8. Simmondsia oil: Simmondsia oil is derived from the seeds and other parts of the jojoba plant. It closely mimics the natural oil of the skin and has antimicrobial and antioxidant properties.
9. Mentha oil: Mentha oil is extracted from the Mentha plant and is commonly known as peppermint oil. It has anti-bacterial and anti-fungal properties.

### BRIEF DESCRIPTION OF DRAWINGS

The present invention will be better understood after reading the following detailed description of the presently preferred aspects thereof with reference to the appended drawings, in which features, other aspects and advantages of certain exemplary embodiments of the invention will be more apparent from the accompanying drawings in which:

FIG. 1 represents the flow diagram of the nanoemulsion-based nanocream product wherein the primary stage is to prepare the nanoemulsion and then replace the water in the aqueous phase of the nanocream product with the nanoemulsion during the formulation to obtain a topical formulation with significantly improved performance characteristics.

### DETAILED DESCRIPTION

The following description describes various features and functions of the disclosed device and methods with reference to the accompanying figures. In the figures, similar symbols identify similar components, unless context dictates otherwise. The illustrative aspects described herein are not meant to be limiting. It may be readily understood that certain aspects of the disclosed system, method and apparatus can be arranged and combined in a wide variety of different configurations, all of which are contemplated herein.

These and other features and advantages of the present invention may be incorporated into certain embodiments of the invention and will become more fully apparent from the following description and claims or may be learned by the practice of the invention as set forth hereinafter.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings but are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a,” “an,” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

In accordance with the present invention, the nanoemulsion is developed by low-energy emulsification such as the Phase Inversion Temperature (PIT) method which harnesses the system's internal chemical energy. The method helps lower the preparation loss, promotes sustainability, and reduces the risk of damage to sensitive compounds like proteins and peptides.

In accordance with the present invention a nanoemulsion for the formulation of a nanocream product comprising an aqueous phase, an oil phase comprising oil or a blend of oils having one or more hydrophobic active ingredients, a primary surfactant system having at least one ionic and at least non-ionic part, and a co-surfactant system wherein glycerine, cetosterearyl alcohol, and glyceryl monostearate act as stabilizers.

In accordance with the present invention, the formulation of the present invention can be used to make emollient products. An emollient product is referred to as a substance that softens and moisturizes the skin upon topical application. It works by forming a protective layer on the surface of the skin, preventing moisture loss. The emollients extend themselves in various forms including lotions, creams, ointments, oils, butter, gels, barrier creams, and balms.

Further, in the first embodiment, the blend of oils in the oil phase comprises 10-50% of tocopherol, 10-50% cinnamomum oil, 10-50% simmondsia oil, 10-50% mentha oil; the primary surfactant including but not limited to Tween 80, Tween 20, sorbitan monooleate, polyglycerol esters, ocamidopropyl betaine; and the co-surfactant including but not limited to glycerine, cetosterearyl alcohol, and glyceryl monostearate.

### Development of Nanoemulsion System by Phase Inversion Temperature (PIT) Method:

For the preparation of about 10 grams of the nanoemulsion using the Phase Inversion Temperature (PIT) method includes selecting the oil phase consisting of a blend of 10-50% of tocopherol, 10-50% cinnamomum oil, 10-50% simmondsia oil, 10-50% mentha oil; the primary surfactant including but not limited to Tween 80, Tween 20, sorbitan monooleate, polyglycerol esters, cocamidopropyl betaine; and the co-surfactant including but not limited to glycerine, cetosterearyl alcohol, and glyceryl monostearate.

The combined oil phase and the surfactant were weighed to 1.2 grams (12% of the total formulation). Additionally, 3.5% glycerine (0.35 grams), 2% cetostearyl alcohol (0.2 grams), and 2.5% glyceryl monostearate (0.25 grams) were included as co-surfactants to further stabilize the nanoemulsion.

To begin the formulation, the oil phase was mixed with the surfactant and co-surfactants in a glass vial and heated to a temperature of 10-30° C. above the PIT. In a separate vial, 50-80% of the total distilled water (8 grams) was also heated to 10-30° C. above the PIT. Once both phases were preheated, the water phase was added to the heated oil and surfactant mixture while stirring continuously at 50-300 revolutions per minute (RPM), with the system maintained at an elevated temperature. The stirring was continued for 5-15 minutes, during which the system appeared turbid and inhomogeneous. Following this, the entire system was rapidly cooled to 15-25° C. by placing the glass vial in ice-cold water, inducing temperature quenching. The stirring was maintained during this cooling process to ensure proper emulsification, leading to the formation of a transparent and stable nanoemulsion.

However, preferably, the oil phase used consists of a blend of 30% vitamin E, 30% cinnamon oil, 20% jojoba oil, and 20% peppermint oil, while Tween 80 was chosen as the primary surfactant. To begin the formulation, the oil phase was mixed with the surfactant and co-surfactants in a glass vial and heated to a temperature of 10° C. above the PIT. In a separate vial, 80% of the total distilled water (8 grams) was also heated to 10° C. above the PIT. Once both phases were preheated, the water phase was added to the heated oil and surfactant mixture while stirring continuously at 300 rpm, with the system maintained at an elevated temperature. Stirring was continued for 10 minutes, during which the system appeared turbid and inhomogeneous. Following this, the entire system was rapidly cooled to 25° C. by placing the glass vial in ice-cold water, inducing temperature quenching. Stirring was maintained during this cooling process to ensure proper emulsification, leading to the formation of a transparent and stable nanoemulsion.

To produce a nanoemulsion using the Phase Inversion Temperature (PIT) method, the oil-in-water O/W emulsion system is heated until the phase inversion occurs. Subsequently, the emulsion system is rapidly cooled with continuous stirring, a process called temperature quenching. This cooling reverses the phase inversion, transforming the system back to an oil-in-water O/W emulsion. The turbulence caused by the rapid temperature change promotes the formation of nano-sized oil droplets, resulting in a stable oil-in-water O/W nanoemulsion.

A total of 9 nanoemulsion systems with varying ratios of oil phase and surfactant were prepared and tested to achieve the best nanoemulsion with details provided in the table.

TABLE 1
Formulation batches of nanoemulsions

| | Oil | Tween | 3.5% | 2.5% | 2% | 80% | |
|---|---|---|---|---|---|---|---|
| | phase | 80 | Glycerine | GMS | CSA | Water | Total |
| NE code | (gm) | (gm) | (gm) | (gm) | (gm) | (8 gm) | (gm) |
| NE1 | 0.72 | 0.48 | 0.35 | 0.25 | 0.2 | 8 | 10 |
| NE2 | 0.66 | 0.54 | 0.35 | 0.25 | 0.2 | 8 | 10 |
| NE3 | 0.6 | 0.6 | 0.35 | 0.25 | 0.2 | 8 | 10 |
| NE4 | 0.54 | 0.66 | 0.35 | 0.25 | 0.2 | 8 | 10 |
| NE5 | 0.48 | 0.72 | 0.35 | 0.25 | 0.2 | 8 | 10 |
| NE6 | 0.45 | 0.75 | 0.35 | 0.25 | 0.2 | 8 | 10 |
| NE7 | 0.42 | 0.78 | 0.35 | 0.25 | 0.2 | 8 | 10 |
| NE8 | 0.39 | 0.81 | 0.35 | 0.25 | 0.2 | 8 | 10 |
| NE9 | 0.36 | 0.84 | 0.35 | 0.25 | 0.2 | 8 | 10 |

The notations in the table are as follows NE=Nanoemulsions, CSA=Cetostearyl Alcohol, GMS=Glyceryl Monostearate.

In this method, polyethoxylated surfactants, such as Tween 80, which are hydrophilic at lower temperatures, typically form oil-in-water (O/W) emulsions. However, when the emulsion system is heated, the polyoxyethylene groups in these surfactants undergo dehydration, making the surfactants more lipophilic. As a result, the spontaneous curvature of the surfactant molecules shifts, leading to the formation of a water-in-oil (W/O) emulsion. The temperature at which this phase inversion occurs is referred to as the Phase Inversion Temperature (PIT), also known as the HLB (Hydrophilic-Lipophilic Balance) temperature.

### Evaluation of the Developed Nanoemulsion System:

### a) Thermodynamic Stability Testing

The thermodynamic stability of the nanoemulsion formulations was evaluated through centrifugation and heating-cooling (H/C) cycle tests. In the centrifugation test, each formulation was subjected to a centrifuge at 3500 rpm for 15 minutes. After centrifugation, the formulations were visually inspected for any signs of instability, such as phase separation or creaming, which would indicate poor stability.

For the heating-cooling cycle test, the formulations were alternately exposed to temperatures of 4° C. and 45° C., with each cycle lasting 24 hours at each temperature. After completing the cycles, the formulations were examined for any changes in stability, including phase separation, turbidity, or other signs of instability. These tests were conducted to ensure the nanoemulsions maintained their structural integrity under varying conditions.

### b) Determination of Percentage Transmittance for Transparency of the System

The transparency of the nanoemulsion formulations was assessed by determining the percentage of transmittance (% transmittance), which provides an indication of droplet size within the nanometer range. To measure % transmittance, all formulations were first diluted 50 times with distilled water to ensure accurate readings. The diluted samples were then analyzed using a UV spectrophotometer set at a wavelength of 650 nm, with distilled water used as a blank reference.

### c) Mean Droplet Size (MDS) and Polydispersity Index (PDI) Determination of Optimum Nanoemulsion System

The mean droplet size (MDS) and polydispersity index (PDI) of the optimum nanoemulsion system were determined using the dynamic light scattering (DLS) technique with a Zetasizer (Malvern Instruments). To prepare the sample for measurement, the nanoemulsion formulation was diluted 20 times (1:20) with distilled water to ensure accurate detection of droplet size distribution. The diluted sample was then analyzed at a scattering angle of 90° and a temperature of 25° C. using the Zetasizer. This method provided detailed information on the average droplet size and the uniformity of the droplet size distribution (PDI), which are critical parameters for assessing the stability and performance of the nanoemulsion.

### Development of Nanoemulsion-Based Nanocream Product

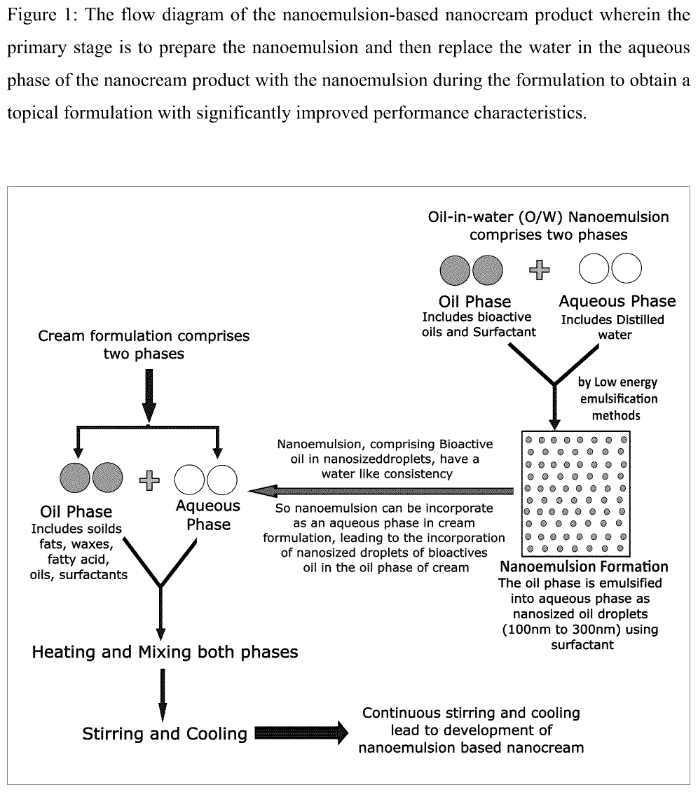

The nanocream product is developed using a novel approach by incorporating nanoemulsion as the aqueous phase in the cream formulation as shown in FIG. 1. Nanoemulsion, with its water-like consistency, was used in place of water, improving the stability of nanosized droplets and reducing the risk of coalescence of bioactive oils. This is due to the highly viscous and thick nature of the cream's oily base, which restricts the movement of nanoemulsion droplets, preventing them from merging.

In accordance with the present invention, the formulated oil-in-water nanoemulsion replaces the water in the aqueous phase during the formulation of the nanocream product. The replacement of the water from the nanoemulsion in the aqueous phase of the nanocream improves the stability of the nano-sized droplets and reduces the risk of coalescence. For the oil phase of the nanocream product, natural emulsifiers and waxes including but not limited to beeswax, cocoa butter, lecithin, agar, xanthan gum, palm wax, rice bran wax, candelilla wax, carnauba wax, cetostearyl alcohol, glyceryl monostearate, polyglycerol esters, sorbitan monostearate, stearoyl lactylate, monoglycerides, and diglycerides were utilized as the cream's oily base.

Different ratios of the oil and aqueous phases were used to prepare six nanocream formulations, as shown in Table 2.

For the said preparation, for the oil phase of the nanocream product, beeswax, and cocoa butter were utilized as the cream's oily base, while cetostearyl alcohol and glyceryl monostearate served as emulsifiers. The aqueous phase consisted of the developed optimum nanoemulsion, containing vitamin E, cinnamon oil, jojoba oil, and peppermint oil, along with glycerine as a humectant.

TABLE 2
Formulation batches of nanocream using
nanoemulsion as aqueous phase

| | Oil Phase (%) | | | | | Aqueous phase (%) | | |
|---|---|---|---|---|---|---|---|---|
| | | | | | Total | | | Total |
| NC | | | Coco | Bees | Oil | Glyc- | | Aqueous |
| Code | GMS | CSA | Butter | Wax | Phase | erine | NE | Phase |
| (#) | (%) | (%) | (%) | (%) | (%) | (%) | (%) | (%) |
| NC1 | 9 | 9 | 8 | 14 | 40 | 4 | 56 | 60 |
| NC2 | 8.5 | 8.5 | 7 | 13.5 | 37.5 | 4 | 58.5 | 62.5 |
| NC3 | 8 | 8 | 6 | 13 | 35 | 4 | 61 | 65 |
| NC4 | 7 | 7 | 4 | 12 | 30 | 4 | 66 | 70 |
| NC5 | 6 | 6 | 2 | 11 | 25 | 4 | 71 | 75 |
| NC6 | 5.5 | 5.5 | 1 | 10.5 | 22.5 | 4 | 73.5 | 77.5 |

The notations in the table are as follows NE=Nanoemulsions, CSA=Cetostearyl Alcohol, GMS=Glyceryl Monostearate, NC=Nano Cream (a form of nanocream product)

To begin the development of the nanoemulsion-based nanocream product, the oil phase is prepared by mixing and weighing the oil phase ingredients and emulsifiers and melting the mixture at 65° C. while continuously stirring. The aqueous phase is prepared by combining the formulated nanoemulsion and glycerine and heating the combination to 55° C. below the phase inversion temperature of the nanoemulsion. Thereafter, the pre-heated aqueous phase is gradually added to the pre-heated oil phase with continuous stirring and ultimately cooling the mixture to room temperature and during this step, the cooling process is conducted slowly to ensure a smooth emulsion. The complete addition of the aqueous phase to the oil phase while continuously stirring and cooling leads to a homogeneous and viscous nanocream product.

FIG. 1 of the present invention is a diagrammatic representation of a nanoemulsion-based nanocream product wherein the oil-in-water nanoemulsion comprises two phases i.e., the oil phase and the aqueous phase. The oil phase includes oils, oil-soluble bioactive, and emulsifying agents whereas the aqueous phase contains distilled water. Upon combining both phases, the oil phase gets emulsified into the aqueous phase as nanosized oil droplets (100-300 nm) using surfactants, and a nanoemulsion formulation is obtained. These nanoemulsions have a water-like consistency and can be used in the aqueous phase of cream/nanocream formulations, leading to the incorporation of nano-sized droplets of bioactive oils in the oil phase of the cream.

The nanocream is formed by combining the oil and aqueous phase wherein the oil fats include solid fats, waxes, fatty acids, and other emulsifying agents whereas the aqueous phase comprises the developed nanoemulsions of desired effects. Both mixtures are heated, mixed, stirred, and cooled to obtain a nanocream product with significantly improved efficiency.

### Evaluation of Developed Nanocream Product

### a) Physical Appearance and pH of the Nanocream Product

The organoleptic characteristics of all nanocream formulations were evaluated through visual observation and tactile assessment. The color, texture, homogeneity, and any signs of phase separation were examined by inspecting the appearance of each formulation. To further assess homogeneity and texture, a small amount of the nanocream product was pressed and rubbed between the thumb and index finger. This allowed for the evaluation of immediate skin feel, including sensations of stiffness, grittiness, greasiness, and overall smoothness. For the pH determination, one gram of each nanocream formulation was dispersed in 25 mL of deionized water. The pH of the resulting dispersion was measured using a calibrated pH meter to ensure the formulations were suitable for topical application and within the desired pH range for skin compatibility.

### b) Stability Determination by Centrifugation Test

To evaluate the stability of the oil-in-water (O/W) nanocream formulation against creaming, a centrifugation test was conducted. Creaming is a common instability reaction in O/W emulsions, where the dispersed oil phase may separate and rise to the top, forming a layer of oil droplets. This test helps to confirm that the developed nanocream product is stable, well-emulsified, and homogenous.

For the centrifugation test, 5 grams of the nanocream product, was placed in a centrifuge tube and centrifuged at 3500 rpm for 20 minutes. After centrifugation, the product was carefully inspected for any signs of creaming, phase separation, or visible layers of oil droplets. The absence of these signs would indicate that the nanocream has good stability and resistance to creaming under accelerated conditions.

### c) Spreadability Determination

To determine the spreadability of the nanocream product, a sample weighing 1 gram was placed at the center of a square glass plate (dimensions: 10 cm×20 cm). A second, identical glass plate was carefully placed on top of the nanocream product, and a 1.5 kg weight was applied at the center for 1 minute to allow the nanocream product to spread between the plates. After the designated time, the plates were separated, and the spread diameter of the nanocream product was measured in four different directions: vertical, horizontal, and two diagonals. The average of these four measurements was calculated to obtain the final Spreadability reading, ensuring an accurate representation of the nanocream product's spreading capacity.

### d) In-Vitro Membrane Permeation Studies of the Developed Nanocream Product

For the in-vitro membrane permeation study, the optimum nanocream product formulation was evaluated and compared with the normal cream formulation (C2), where no nanoemulsion was used as the aqueous phase. The permeation study employed the dialysis bag method. A dialysis membrane (Dialysis membrane-150; LA401-5MT; average flat width 42.44 mm and average diameter 25.4 mm, HiMedia Laboratories, Mumbai, India) was activated by immersing it in distilled water for 24 hours. After activation, one end of the dialysis membrane was securely tied with a thread. Five grams of the nanocream product formulation were then loaded into the dialysis membrane through the other end, which was subsequently tied to ensure no leakage occurred. The sealed dialysis bag, containing the nanocream product, was submerged in 500 mL of distilled water at a controlled temperature of 37° C. The system was stirred continuously at 200 rpm. At specified time intervals (30 min, 1 h, 2 h, 3 h, etc.), 5 mL samples were withdrawn from the beaker. After each withdrawal, an equivalent volume of distilled water was added to maintain sink conditions. The collected samples were filtered, diluted with methanol, and analyzed for the permeation of vitamin E using a UV spectrometer at 290 nm, calibrated against a standard curve of vitamin E in methanol.

### e) Long-Term Stability Testing of the Developed Nanocream Product.

To evaluate the long-term stability of the optimum nanocream product formulation, the product was subjected to a storage stability study. The formulated nanocream product was carefully packed in a glass beaker and stored at room temperature (25±2° C.) for a period of six months.

Over this duration, any potential instability mechanisms were monitored, including changes in physical appearance, spreadability, pH, and phase separation. After the six-month storage period, the nanocream product was reevaluated for these parameters.

Physical appearance was visually inspected to note any alterations in texture, color, or homogeneity. Phase separation was assessed by observing any distinct layers that may have formed, indicating instability. The pH of the formulation was measured by dispersing a small sample in deionized water and using a calibrated pH meter.

Lastly, spreadability was determined using a glass plate method to quantify any changes in the ease of application compared to its initial state. These tests provided valuable insights into the formulation's ability to retain its original properties over time, ensuring its long-term stability and effectiveness.

### Experiments and Results

### Development of UV-Spectrophotometric Method for Estimation of Bioactive Oil

In the calibration curve of vitamin E in methanol, linearity ranges from 10 to 100 μg/mL methanol with a correlation coefficient (r) of 0.996. The regression equation was found to be Y=0.0062x+0.1995.

### Evaluation and Selection of Optimum Nanocream Formulation

A total of nine nanocream products were developed by varying the ratios of the oil phase and surfactant. These formulations were evaluated based on their thermodynamic stability and transparency to select the optimum nanoemulsion system for further use in nanocream product formulation.

The results of the thermodynamic stability tests and the transparency evaluation are presented in the table 3. Among the nine formulations, NE9, NE8, NE7, and NE6 exhibited a transparent to translucent appearance, indicative of nanosized droplets.

NE6 was selected as the optimum nanoemulsion formulation due to its superior composition, which included a higher proportion of bioactive oils and a lower surfactant concentration compared to NE9, NE8, and NE7. Additionally, NE6 demonstrated thermodynamic stability as well.

Further evaluation of NE6 revealed a mean droplet size (MDS) of 121 nm and a polydispersity index (PDI) of 0.094. The nano range droplet size confirms its classification as a nanoemulsion, while the relatively low PDI value reflects a high degree of mono-dispersity, indicating uniform droplet size distribution. This uniformity is a desirable characteristic of the nanoemulsion system.

Thus, the NE6 nanoemulsion formulation was chosen as the optimum nanoemulsion and further used for the formulation of nanocream products.

TABLE 3
Result of Evaluation of nanoemulsion formulation

| | | | Thermodynamic | |
|---|---|---|---|---|
| | | | stability testing | |
| NE | | (%) Transmittance | H/C cycle | centrifugation |
| code | Appearance | (Mean ± SD) | tests | test |
| NE1 | Turbid | 4.67 ± 1.22 | Phase | Phase |
| | | | separation | separation |
| NE2 | Turbid | 3.75 ± 1.89 | Phase | Phase |
| | | | separation | separation |
| NE3 | Turbid | 7.71 ± 2.05 | Phase | No phase |
| | | | separation | separation |
| NE4 | Turbid | 20.26 ± 2.88 | Stable | Stable |
| NE5 | Turbid | 24.15 ± 2.91 | Stable | Stable |
| NE6 | Translucent- | 84.74 ± 1.88 | Stable | Stable |
| | transparent | | | |
| NE7 | Translucent- | 92.15 ± 1.05 | Stable | Stable |
| | transparent | | | |
| NE8 | Transparent | 98.17 ± 0.93 | Stable | Stable |
| NE9 | Transparent | 98.03 ± 0.76 | Stable | Stable |

The notations used in the table are as follows:
n=3; SD: standard deviation; H/C cycle: heating cooling cycle.

### Evaluation and Selection of Optimum Nanocream Products' Formulation

The results of the evaluation of all six nanocream product formulations are presented in the table 4. Each formulation exhibited good emulsification and homogeneity, with no signs of grittiness. The successful emulsification was further supported by the centrifugation test, in which all formulations demonstrated stability, with no phase separation. The pH values of all nanocream products ranged within the normal skin pH range, suggesting minimal risk of skin irritation upon application. It was observed that formulations with an oil phase exceeding 30% and an aqueous phase below 65% were thick and hard in texture. Additionally, a slight increase in pH was noted as the total oil 5 phase increased.

Among all formulations, NC4 has optimal spreadability, which would enhance ease of application on the skin. Based on this evaluation, NC4 was selected as the optimum nanocream product formulation for spreadability.

In NC4, the total nanocream product used as the aqueous phase was 66%, allowing for the incorporation of significant quantities of bioactive oils: 891 mg of vitamin E, 891 mg of cinnamon oil, 594 mg of jojoba oil, and 594 mg of peppermint oil as nanosized droplets (in 66 gm of total nanoemulsion as aqueous phase) in 100 grams of nanocream product.

TABLE 4
Result of Evaluation of nanocream product formulation

| | | | Centri- | Spread- |
|---|---|---|---|---|
| NC | | pH | fugation | ability |
| code no | Appearance | (Mean ± SD); | test | (mm) |
| NC1 | Dense, thick creams | 6.05 ± 0.12 | Stable | 40.5 |
| | with a firm | | | |
| | consistency | | | |
| NC2 | Slight Dense, thick | 5.95 ± 0.09 | Stable | 46.25 |
| | creams with a | | | |
| | firm consistency | | | |
| NC3 | Slight Dense, thick | 5.98 ± 0.06 | Stable | 54.75 |
| | creams with a firm | | | |
| | consistency | | | |
| NC4 | Light, smooth creams, | 5.56 ± 0.15 | Stable | 65.5 |
| | and soft consistency | | | |
| NC5 | Light, smooth creams, | 5.35 ± 0.20 | Stable | 72.5 |
| | and soft consistency | | | |
| NC6 | Creamy, fluid-like | 5.31 ± 0.06 | Stable | 88.25 |
| | consistency, more | | | |
| | liquid in nature | | | |

| | | |
|---|---|---|
| Commercial | Monange ® - Savoy Industria de Cosmeticos, | 62 ± 75 |
| Cream | Goiania, Brazil (Moisturizing cream) | |

The notations used in Table 4 are as follows: n=3; SD: standard deviation.

### Effect of Nanoemulsion as an Aqueous Phase in Nanocream Products' Formulation

To evaluate the impact of incorporating nanoemulsion as the aqueous phase in the nanocream product formulation, two additional cream formulations were prepared using the composition of the optimum nanocream formulation (NC4), as detailed in table 5. The evaluation results are also presented in the table 6.

When water alone was used as the aqueous phase, the formulation failed to emulsify, resulting in an unstable cream that could not form properly. This indicates that Cetostearyl Alcohol (CSA) and Glyceryl Monostearate (GMS) alone were insufficient to emulsify 70% of the aqueous phase in the cream.

However, when 5% Tween 80 was added along with water in the C2 formulation, the cream was emulsified successfully, though it lacked homogeneity and displayed small, soft lumps compared to the smoother NC4 formulation. This demonstrates that the presence of Tween 80 in the nanoemulsion not only reduced the size of bioactive oil droplets to the nanoscale but also facilitated the emulsification of two phases of cream, contributing to a smoother and more homogenous cream.

Thus, the use of nanoemulsion as the aqueous phase, with Tween 80 playing a dual role, significantly improves the texture, emulsification, and overall quality of the nanocream product's formulation.

TABLE 5
Cream formulation by using water as aqueous phase

| | Oil Phase (%) | | | | | | Aqueous phase (%) | | |
|---|---|---|---|---|---|---|---|---|---|
| | | | | | | Total | | | Total |
| Cream | | | Coco | Bees | Tween | Oil | | | Aqueous |
| Code | GMS | CSA | Butter | Wax | 80 | Phase | Glycerine | Water | Phase |
| (#) | (%) | (%) | (%) | (%) | (%) | (%) | (%) | (%) | (%) |
| C1 | 7 | 7 | 4 | 12 | — | 30 | 4 | 66 | 70 |
| C2 | 7 | 7 | 4 | 12 | 5 | 35 | 4 | 61 | 65 |

The notations used in the table are as follows: CSA, Cetostearyl Alcohol; GMS, and Glyceryl Monostearate.

Overall, the NC4 nanocream formulation was selected as the optimum formulation based on its superior emulsification, homogeneity, and spreadability. It was further evaluated through in-vitro membrane permeation studies, and long-term stability, to confirm its enhanced performance and potential for therapeutic effectiveness.

TABLE 6
Result of Evaluation of cream formulation.

| | | pH | Centri- | Spreada- |
|---|---|---|---|---|
| | | (Mean ± SD); | fugation | bility |
| C code no | Appearance | n = 3 | test | (mm) |
| C1 | Not emulsified, | — | — | — |
| | unstable | | | |
| C2 | Light cream soft | 5.06 ± 0.16 | Stable | 75.25 |
| | consistency | | | |
| | with lumps, | | | |
| | not homogenous | | | |

### In-Vitro Membrane Permeation Studies of NC4 Nanocream Formulation.

The results of the in-vitro membrane permeation study comparing the optimum nanocream formulation (NC4) with the normal cream formulation (C2) are illustrated in the FIGURE. It was observed that the permeation of bioactive oils through the dialysis membrane was significantly higher for the nanocream (NC4) than for the normal cream (C2). At the end of the 10-hour study, the NC4 nanocream demonstrated a permeation rate of 97.15±2.33% (Mean±SD; n=3), whereas the C2 cream showed only 62.46±2.18% (Mean±SD; n=3) permeation.

This substantial difference in permeation between the two formulations can be attributed to the nanosized droplets in the NC4 nanocream, which allow for more efficient penetration through the membrane. These findings suggest that the nanoscale structure of the nanocream significantly improves its delivery potential, pointing to enhanced therapeutic effectiveness in comparison to conventional cream formulations. The superior in-vitro permeation performance of the NC4 nanocream indicates its potential for better skin absorption and efficacy in practical applications.

### Long-Term Stability Testing of NC4 Nanocream Formulation

The long-term stability of the selected optimum nanocream formulation NC4 was assessed after storing it at room temperature (25±2° C.) for a period of six months. The results of the stability study, summarized in Table 7, demonstrated that the formulation maintained its stability throughout the six-month duration.

Notably, there were no signs of phase separation, and the nanocream formulation Nc4 exhibited a homogeneous appearance. Furthermore, measurements indicated that there were no significant changes in pH, suggesting that the formulation's integrity was preserved. Additionally, the spreadability of the nanocream formulation remained consistent with the initial measurements. These findings collectively indicate that the novel nanoemulsion-based nanocream formulation is stable for long-term storage, affirming its potential for practical applications in skin care.

TABLE 7
Long-term stability testing of optimum
nanocream formulation NC4

| | | | Centri- | Spreada- |
|---|---|---|---|---|
| NC | | pH | fugation | bility |
| code no | Appearance | (Mean ± SD) | test | (mm) |
| Initial | Light, smooth creams, | 5.56 ± 0.15 | Stable | 65.5 |
| | and soft consistency | | | |
| After 6 | Light, smooth creams, | 5.23 ± 0.26 | Stable | 62.25 |
| months | and soft consistency | | | |

The notations used in Table 7 are as follows: n=3; SD: standard deviation

1. A nanoemulsion-based nanocream product from green ingredients exhibiting enhanced performance characteristics, comprising:
an aqueous phase consisting essentially of the nanoemulsion consisting of:
a) an oil phase having a blend of oils wherein the blend of oil has 10-50% of tocopherol, 10-50% of cinnamomum oil, 10-50% of simmondsia oil, 10-50% of mentha oil;
b) a primary surfactant such as Tween 80, Tween 20, sorbitan monooleate, polyglycerol esters, cocamidopropyl betaine and mixtures thereof wherein, the surfactant has at least one ionic and at least one non-ionic part;
c) a co-surfactant such as glycerine in the range of 0-5.5%, cetostearyl alcohol in the range of 0-5%, and glyceryl Monostearate in the range of 0-4% to improve the stability;
an oil phase consisting of natural emulsifiers and waxes such as beeswax, cocoa butter, lecithin, agar, xanthan gum, palm wax, rice bran wax, candelilla wax, carnauba wax, cetostearyl alcohol, glyceryl monostearate, polyglycerol esters, sorbitan monostearate, stearoyl lactylate, monoglycerides, diglycerides and mixtures thereof to develop an oily nanocream base.

2. The nanoemulsion-based nanocream product from green ingredients exhibiting enhanced performance characteristics as claimed in claim 1 wherein, the water-like consistency of the nanoemulsion replaces the water in the nanocream product's aqueous base so that the stability of the nano-sized droplets is improved.

3. The nanoemulsion-based nanocream product from green ingredients exhibiting enhanced performance characteristics as claimed in claim 1 wherein, the blend of oils in the oily phase of nanoemulsion consists of 30% of tocopherol, 30% of cinnamomum oil, 30% of simmondsia oil, and 30% of mentha oil.

4. The nanoemulsion-based nanocream product from green ingredients exhibiting enhanced performance characteristics as claimed in claim 1 wherein, the quantity of co-surfactants in the nanoemulsion is 3.5% glycerine, 2% cetostearyl alcohol, and 2.5% glyceryl monostearate.

5. The nanoemulsion-based nanocream product from green ingredients exhibiting enhanced performance characteristics as claimed in claim 1 wherein, the utilization of nanoemulsion in the aqueous phase of the nanocream product reduces the risk of coalescence of nano sized droplets of bioactive oils.

6. The nanoemulsion-based nanocream product from green ingredients exhibiting enhanced performance characteristics as claimed in claim 1 wherein, the movement of the nanoemulsion droplets in the nanocream product is restricted due to the thick and viscous nature of the oil-based base of the nanocream product.

7. The nanoemulsion-based nanocream product from green ingredients exhibiting enhanced performance characteristics as claimed in claim 1 wherein, the formulated nanocream product when rubbed between the fingers exhibits immediate skin feel, smoothness and no sign of grittiness.

8. The nanoemulsion-based nanocream product from green ingredients exhibiting enhanced performance characteristics as claimed in claim 1 wherein, about one gram of the nanocream product is dispersed in about 25 ml of deionized water to measure the pH, ensuring the formulation is compatible for topical application on the skin.

9. The nanoemulsion-based nanocream product from green ingredients exhibiting enhanced performance characteristics as claimed in claim 1 wherein, the pH of the dispersion of nanocream product in deionized water, is measured using a calibrated pH meter to ensure skin compatibility.

10. The nanoemulsion-based nanocream product from green ingredients exhibiting enhanced performance characteristics as claimed in claim 1 wherein, the formulated nanocream product is an oil-in-water (O/W) composition.

11. The nanoemulsion-based nanocream product from green ingredients exhibiting enhanced performance characteristics as claimed in claim 1 wherein, the stability of the formulated nanocream product is validated by the centrifugation test whereby about 5 grams of nano-cream is centrifuged at about 1000-3500 revolutions per minute for about 20-30 minutes;
wherein, post-centrifugation, the nanocream product is inspected to ensure that it lacks the signs of creaming, phase separation, or any visible layers of oil droplets thereby corroborating stability.

12. The nanoemulsion-based nanocream product from green ingredients exhibiting enhanced performance characteristics as claimed in claim 1 wherein, the spreadability of the formulated nanocream product is validated by placing two square glass plates on top of each other whereby about 1.5 kg weight was applied at the center for about 1 minute to allow the nanocream product to spread between the plates and the spread diameter of the nanocream product is measured in four different directions including vertical, horizontal, and two diagonals;
wherein, the average of these four measurements is calculated to obtain the final spreadability reading, ensuring an accurate representation of the spreading capacity.

13. A method to formulate the nanoemulsion-based nanocream product from green ingredients exhibiting enhanced performance characteristics, comprising the steps of:
Mixing, the oil phase with the primary surfactant in a glass vial and heating it 10-30° C. above the phase inversion temperature;
Heating, 50-80% of the total distilled water above 10-30° C. above the phase inversion temperature in a separate vial;
Adding, the water phase to the heated oil phase and surfactant mixture while stirring continuously for 50-300 revolutions per minute wherein the system is maintained at the elevated temperature;
Stirring, continuously for 5-15 minutes to obtain an inhomogeneous and turbid solution followed by rapid cooling of the glass vial at 15-25° C. in ice cold water thereby inducing temperature quenching;
Emulsifying, the solution by continuous stirring during the cooling process to achieve a transparent and stable nanoemulsion;
Mixing and weighing the oil phase ingredients and emulsifiers of the cream base and melting the mixture at 40-70° C. while continuously stirring;
Heating, the aqueous phase comprising formulated nanoemulsion and glycerine at 35-55° C. below the phase inversion temperature of the formulated nanoemulsion;
Adding, the pre-heated aqueous phase gradually to the pre-heated oil phase with continuous stirring and ultimately cooling the mixture to room temperature;
wherein the cooling process is conducted gradually to ensure a smooth emulsion;
wherein, the complete addition of the aqueous phase to the oil phase while continuously stirring and cooling leads to a homogeneous and viscous nanocream product.

14. The method to formulate the nanoemulsion-based nanocream product from green ingredients exhibiting enhanced performance characteristics as claimed in claim 13 wherein, the method utilizes a low energy mechanism thus preserving the properties and integrity of the bioactive oils potentially leading to enhanced therapeutic effect.

15. The method to formulate the nanoemulsion-based nanocream product from green ingredients exhibiting enhanced performance characteristics as claimed in claim 13 wherein, the heating range of the primary surfactant and distilled water is 10° C. above the phase inversion temperature.

16. The method to formulate the nanoemulsion-based nanocream product from green ingredients exhibiting enhanced performance characteristics as claimed in claim 13 wherein, the low energy method used to formulate the nanoemulsion-based nanocream product exhibiting enhanced performance characteristics harnesses the internal chemical energy of the system thereby requiring minimal energy input.

17. The method to formulate the nanoemulsion-based nanocream product from green ingredients exhibiting enhanced performance characteristics as claimed in claim 13 wherein, the low energy method used to formulate the nanoemulsion-based nanocream product exhibiting enhanced performance characteristics, leads to lower preparation costs and promotes sustainability.

18. The method to formulate the nanoemulsion-based nanocream product from green ingredients exhibiting enhanced performance characteristics as claimed in claim 13 wherein, the low energy method used to formulate the nanoemulsion-based nanocream product exhibiting enhanced performance characteristics, mitigates the risk of damage to sensitive compounds during the formulation.

19. The method to formulate the nanoemulsion-based nanocream product from green ingredients exhibiting enhanced performance characteristics as claimed in claim 13 wherein, the low energy method used to formulate the nanoemulsion-based nanocream product exhibiting enhanced performance characteristics, avoids poor productivity and heat-induced deterioration of the bioactive compounds.

20. The method to formulate the nanoemulsion-based nanocream product from green ingredients exhibiting enhanced performance characteristics as claimed in claim 13 wherein, in the low energy method the polyoxyethylene groups in the surfactants undergo dehydration upon heating thereby making the surfactants more lipophilic.

21. The method to formulate the nanoemulsion-based nanocream product from green ingredients exhibiting enhanced performance characteristics as claimed in claim 13 wherein, in low energy method due to the enhanced lipophilic nature of the surfactants at high temperature the spontaneous curvature of the surfactant molecules shifts leading to the formation of a water-in-oil (W/O) emulsion.

22. The method to formulate the nanoemulsion-based nanocream product from green ingredients exhibiting enhanced performance characteristics as claimed in claim 13 wherein, in low energy method the oil-in-water (O/W) emulsion system is heated until the phase inversion occurs followed by rapid cooling.

23. The method to formulate the nanoemulsion-based nanocream product from green ingredients exhibiting enhanced performance characteristics as claimed in claim 13 wherein, the rapid cooling reverses the phase inversion and transforms the emulsion system back to an oil-in-water (O/W) emulsion thereby promoting the formation of stable nano-sized oil droplets and thus the nanoemulsion.

24. The method to formulate the nanoemulsion-based nanocream product from green ingredients exhibiting enhanced performance characteristics as claimed in claim 13 wherein, the optimum nanoemulsion formulation exhibited a mean droplet size (MDS) of about 121 nm.

25. The method to formulate the nanoemulsion-based nanocream product from green ingredients exhibiting enhanced performance characteristics as claimed in claim 13 wherein, the optimum nanoemulsion formulation exhibited a polydispersity index (PDI) of about 0.094.

26. The method to formulate the nanoemulsion-based nanocream product from green ingredients exhibiting enhanced performance characteristics as claimed in claim 13 wherein the presence of Tween 80 in the nanoemulsion reduces the size of bioactive oil droplets.

27. The method to formulate the nanoemulsion-based nanocream product from green ingredients exhibiting enhanced performance characteristics as claimed in claim 13 wherein the presence of Tween 80 in the nanoemulsion facilitates the emulsification of two phases of cream, contributing to a smoother and more homogenous cream.

28. The method to formulate the nanoemulsion-based nanocream product from green ingredients exhibiting enhanced performance characteristics as claimed in claim 13 wherein, the use of nanoemulsion as the aqueous phase, with Tween 80 playing a dual role, reduces the size of bioactive oil droplets to nanoscale and significantly improves the texture, emulsification, and overall quality of the nanocream formulation.
